(19) United States
(12) Patent Application Publication
(10) Pub. No.: US 2012/0009258 A1
(43) Pub. Date: Jan. 12, 2012

(54) COMPACTED CINACALCET

(75) Inventors: Kathrin Rimkus, Iserlohn (DE); Frank Muskulus, Laupheim (DE); Sandra Brueck, Ottenhofen (DE); Jana Paetz, Bonn (DE)

(73) Assignee: RATIOPHARM GMBH, Ulm (DE)

(21) Appl. No.: 13/120,889

(22) PCT Filed: Sep. 24, 2009
(86) PCT No.: PCT/EP2009/006907

§ 371 Date: Sep. 20, 2011

(30) Foreign Application Priority Data
Sep. 25, 2008 (DE) 10 2008 048 836.4

(51) Int. Cl. A61K 31/137; A61P 5/18; A61K 9/28

(52) U.S. Cl. 424/465; 514/655

(57) ABSTRACT

The invention relates to an intermediate, obtainable by jointly compacting (i) crystalline cinacalcet or a pharmaceutically acceptable salt thereof, with (ii) a hydrophilising agent, and also tablets containing the intermediates of the invention. The invention further relates to cinacalcet tablets with a bimodal pore size distribution and a method of preparing the tablets of the invention. Finally, the invention relates to the use of a pH adjuster for preparing cinacalcet formulations which can preferably be administered independently of mealtimes.

The invention relates to an intermediate obtainable by jointly compacting (i) crystalline cinacalcet or a pharmaceutically acceptable salt thereof, with (ii) a hydrophilising agent, and tablets containing the intermediates of the invention. The invention further relates to cinacalcet tablets with a bimodal pore size distribution and a method of preparing the tablets of the invention. Finally, the invention relates to the use of a pH adjuster for preparing cinacalcet formulations which can preferably be administered independently of mealtimes.

N-[(1R)-1-(1-naphthyl)ethyl]-3-[3-(trifluoromethyl)phenyl]propane-1-amine is known by the INN name “cinacalcet” and has the following structural formula:

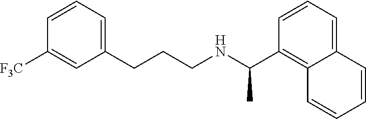

Cinacalcet is a calcimimetic which is used to treat secondary hyperparathyroidism as a consequence of chronic renal failure. In addition, the substance is approved for the treatment of hypercalcaemia in patients with parathyroid carcinoma.

The synthesis and effect of cinacalcet are described in EP 1 203 761 B1. Patients with a chronic kidney disease often suffer from a parathyroid hyperfunction (secondary hyperparathyroidism) as a consequence of their underlying disease. Failing kidneys excrete less phosphate with the urine and form less active vitamin D3, which is needed in order to maintain a physiological level of calcium ions in the blood. When the level of calcium ions drops, an increased amount of parathyroid hormone is secreted by the parathyroid glands. Overproduction of parathyroid hormone in turn causes calcium ions to be mobilised from the bones and the bones to become more brittle. Cinacalcet binds to the calcium-sensitive receptors on the surface of the parathyroid cells. As a result, the sensitivity of the receptor to extracellular calcium ions is enhanced and simulates a higher calcium level in the blood than is actually present. As a result, the secretion of parathyroid hormone drops, and consequently less calcium is released from the bones.

Cinacalcet is also available in amorphous form by spray-drying, cf. WO 2008/000422 A1. Active agents in amorphous form, however, frequently have disadvantageous properties with regard to their storage stability.

WO 2008/064202 describes compositions containing cinacalcet with delayed release. Dosage forms with delayed release are usually employed for special applications. For a large number of applications, however, dosage forms with immediate release are desirable.

The film-coated tablets currently on the market are tablets with immediate release (=immediate-release tablets) and are described in WO 2005/034928. The tablets contain cinacalcet in micronised form with a proportion of active agent of about 18%. The film-coated tablets should be taken with or shortly after meals, since the bioavailability is increased by 50 to 80 percent when taken at the same time as food and is only then acceptable.

The micronisation of cinacalcet entails a number of disadvantages, however. First of all, the micronisation results in an active agent with undesirably poor flowability. In addition, the micronised active agent is more difficult to compress, and there is occasionally an uneven distribution of the active agent within the pharmaceutical formulation to be compressed. The considerable enlargement of the surface area during micronisation also causes the sensitivity of the active agent to oxidation to increase.

The objective of the present invention was therefore to overcome the above-mentioned disadvantages. The intention is to provide the active agent in a form which possesses good flowability and makes good compression possible. In addition, it is intended to enable an even distribution of the active agent. It is intended to avoid micronisation of the active agent.

The intention is also to provide the active agent in a form which possesses good solubility, with good storage stability at the same time. Even after storage for 2 years (or storage for 3 months under stress conditions), correspondingly good solubility ought to be achievable. The intention is to make administration independently of mealtimes possible. In particular, the aim is to achieve a solubility of greater than 3 mg/ml, especially 10 mg/ml. In addition, it is intended to achieve a storage stability of 12 months at 40° C. and 75% air humidity. The impurities after storage under these conditions are intended to be <2% by weight, especially <1% by weight. Furthermore, it is intended to be possible to provide cinacalcet tablets both with a rapid disintegration time and also with advantageous hardness.

Moreover, it is intended that all the above-mentioned advantageous properties should be achievable with a high proportion of active agent (e.g. with contents of active agent of 20%, 30%, 40% and/or 50%). In particular, it is intended that the above-mentioned properties should also be achievable with a high proportion of active agent and at the same time a high “content uniformity”.

It has been possible to solve the problems of the present invention by means of an intermediate which is obtainable by jointly compacting cinacalcet and hydrophilising agent, and by using the intermediate to prepare tablets with immediate release, wherein the tablets exhibit in particular a bimodal pore distribution.

The subject matter of the invention is thus an intermediate obtainable by jointly compacting
(i) crystalline cinacalcet or a pharmaceutically acceptable salt thereof, with
(ii) a hydrophilising agent.

As a matter of principle, the term “cinacalcet” (i) in the context of this application comprises both the “free base” described above and also pharmaceutically acceptable salts thereof. These may be one or more salts, which may also be present in a mixture. “Salt” is understood in this context to mean that the amine group of cinacalcet has been protonated, resulting in the formation of a positively charged nitrogen atom, which is associated with a corresponding counter-anion.

The salts used are preferably acid addition salts. Examples of suitable salts are hydrochlorides, carbonates, hydrogen carbonates, acetates, lactates, butyrates, propionates, sulphates, methane sulphonates, citrates, tartrates, nitrates, sulphonates, oxalates and/or succinates.

In the case of cinacalcet, it is particularly preferable that the pharmaceutically acceptable salt should be cinacalcet hydrochloride. It is likewise particularly preferable that the pharmaceutically acceptable salt should be cinacalcet carbonate. In addition, it is likewise particularly preferable that the pharmaceutically acceptable salt should be cinacalcet methane sulphonate.

The cinacalcet (i) used, preferably the cinacalcet hydrochloride used, is usually a crystalline material which has not been micronised. It is preferable for cinacalcet hydrochloride in the polymorphous form I to be used. The polymorphous form I is disclosed, for example, in WO 2007/62147.

The term “non-micronised cinacalcet” refers in the context of this invention to particulate cinacalcet which generally has an average particle diameter (D50) of 60 to 250 μm, preferably 65 to 200 μm, more preferably 70 to 125 μm, and especially 75 to 110 μm.

The expression “average particle diameter” relates in the context of this invention to the D50 value of the volume-average particle diameter determined by means of laser diffractometry. Specifically, a Malvern Instruments Mastersizer 2000 was used to determine the particle diameter. All the measuring conditions are selected as described on pages 9 and 10 of WO 2005/034928, i.e. wet measurement, 1,750 rpm, Span® 85 as dispersant, evaluation according to the Fraunhofer method. The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

Analogously, the D10 value of the particle diameter is defined as the particle diameter at which 10% by volume of the particles have a smaller diameter than the diameter which corresponds to the D10 value. Similarly, the D90 value of the particle diameter is defined as the particle diameter at which 90% by volume of the particles have a smaller diameter than the diameter which corresponds to the D90 value.

Furthermore, the non-micronised cinacalcet usually has D10 values of 1 to 50 μm, more preferably 1 to 30 μm, and especially 2 to 25 μm. In addition, the non-micronised cinacalcet usually has D90 values of 200 to 800 μm, more preferably 250 to 700 μm, and especially 300 to 600 μm.

Crystalline cinacalcet is usually present in the form of needles. Characterisation by means of the volume-average particle diameter is therefore frequently not specific enough.

It has become apparent that a more precise characterisation of cinacalcet which can advantageously be used, especially with cinacalcet hydrochloride, can be arrived at by describing the specific surface area.

In a preferred embodiment, (i) crystalline cinacalcet or a pharmaceutically acceptable salt thereof with a specific surface area of 0.01 to 12 m²/g, more preferably 0.1 to 8 m²/g, especially 0.1 to 7 m²/g is used.

The specific surface area is determined in the context of this invention in accordance with the gas adsorption method, especially by means of the BET method.

In a preferred embodiment, the cinacalcet (i) used, especially the cinacalcet hydrochloride, has a water content of 0.01 to 0.20% by weight, more preferably 0.02 to 0.10% by weight. The residual water content is determined according to the Karl Fischer method, using a coulometer at 160° C. A Metrohm 831 KF coulometer with a titration cell without a diaphragm is preferably used. Usually, a 20 mg sample of cinacalcet is analysed.

It has been shown that a higher water content would have a negative influence on the flowability and hence, in the case of a high content of active agent (drug load), on the uniformity of the content (content uniformity).

The “hydrophilising agent” (ii) in the context of this invention is generally a substance which is capable of accumulating (chemically or physically) on cinacalcet or salts thereof and increasing the hydrophilicity of the surface.

The hydrophilising agent (ii) may be hydrophilic polymers. This refers to polymers which possess hydrophilic groups. Examples of suitable hydrophilic groups are hydrooxy, amino, carboxy, sulphonate. In addition the hydrophilic polymer to be used for the preparation of the intermediate preferably has a weight-average molecular weight of 1,000 to 150,000 g/mol, more preferably from 2,000 to 90,000 g/mol. The weight-average molecular weight is preferably determined in the context of this application by means of gel permeation chromatography.

It is preferable that the polymers used as the hydrophilising agent should exhibit substantially no emulsifying effect. This means that the hydrophilising agent used should preferably not contain any combination of hydrophilic and hydrophobic groups (especially hydrophobic fatty acid groups). In addition, it is preferable for the intermediate of the invention not to contain any polymers that have a weight-average molecular weight of more than 150,000 g/mol. As a rule, polymers of this kind have an undesirable influence on the dissolution characteristics.

When the polymer used as the hydrophilising agent is dissolved in water in an amount of 2% by weight, the resulting solution preferably has a viscosity of 0.1 to 8 mPa/s, more preferably 0.5 to 7 mPa/s, especially 1 to 6 mPa/s, measured at 25° C. and determined in accordance with Ph. Eur., 6th edition, chapter 2.2.10.

Furthermore, the hydrophilising agent (ii) also includes solid, non-polymeric compounds which preferably contain polar side groups.

The intermediate of the invention may, for example, comprise the following hydrophilic polymers as hydrophilising agents: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); polyvinyl pyrrolidone, polyvinyl alcohol, polymers of acrylic acid and their salts, polyacrylamide, polymethacrylates, vinyl pyrrolidone/vinyl acetate copolymers (such as Kollidon VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the polymers mentioned.

The hydrophilising agent (ii) particularly preferably used is polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol.

Hydrophilising agents (ii) particularly preferably used are co-block polymers of polyethylene glycol and polypropylene glycol, i.e. polyoxyethylene/polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPas, more preferably 500 to 1,500 mPas, especially 800 to 1,200 mPas.

In the context of this invention, it is also possible to use mixtures of the above-mentioned hydrophilising agents. In one possible embodiment, a mixture of, for example, polyvinyl pyrrolidone and polyoxyethylene/polyoxypropylene block polymer is used.

In a preferred embodiment, the intermediate of the invention contains cinacalcet or a pharmaceutically acceptable salt thereof, preferably in non-micronised form, and hydrophilising agent, wherein the weight ratio of active agent (i) to hydrophilising agent (ii) is 5:1 to 1:5, more preferably 3:1 to 1:3, even more preferably 2:1 to 1:2, especially about 1:1.

It is preferable that the type and amount of the hydrophilising agent are selected such that at least 50% of the surface area of the resulting intermediate particles is covered with hydrophilising agent, more preferably at least 60% of the surface area, particularly preferably at least 80% of the surface area, especially at least 95% of the surface area.

In the context of this invention, it is es particularly preferable that cinacalcet (i) and hydrophilising agent (ii) are compacted jointly.

The compacting can be performed in conventional compacting equipment. The compacting is preferably carried out in a roller compacter. The rolling force is preferably 5 to 70 kN/cm, preferably 10 to 60 kN/cm, more preferably 15 to 50 kN/cm, especially 15 to 25 kN/cm.

The compacting conditions are usually selected such that the intermediate of the invention is present in the form of compacted material (flakes), the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The intermediate of the invention is used for preparing a tablet, preferably an immediate-release tablet.

The subject matter of the invention is therefore an immediate-release tablet containing
(α) intermediate of the invention and
(β) pharmaceutical excipients.

These are the excipients (β) with which the person skilled in the art is familiar, especially those which are described in the European Pharmacopoeia.

Examples of excipients (β) used are disintegrants, anti-stick agents, fillers additives to improve the powder flowability, glidants, wetting agents, gelling agents and/or lubricants.

The ratio of active agent to excipients is preferably selected such that the resulting formulations contain

5 to 60% by weight, more preferably 20 to 45% by weight, non-micronised crystalline cinacalcet and
40 to 95% by weight, more preferably 55 to 80% by weight, pharmaceutically acceptable excipients.

In these ratios specified, the amount of hydrophilising agent used to prepare the intermediate of the invention is counted as an excipient. This means that the amount of active agent refers to the amount of non-micronised cinacalcet contained in the finished formulation.

In a preferred embodiment, the tablet of the invention contains 1 to 40% by weight, 5 to 35% by weight, more preferably 10 to 30% by weight, particularly preferably 15 to 25% by weight disintegrant, based on the total weight of the formulation. “Disintegrants” is the term generally used for substances which accelerate the disintegration of a dosage form, especially a tablet, after it is placed in water. Suitable disintegrants are, for example, organic disintegrants such as carrageenan, croscarmellose, sodium carboxymethyl starch, soya polysaccharide and crospovidone. Alternatively, alkaline disintegrants may be used. The term “alkaline disintegrants” means disintegrants which, when dissolved in water, produce a pH level of more than 7.0.

Crospovidone and/or croscarmellose are particularly preferably used as disintegrants, especially in the above-mentioned amounts.

In a preferred embodiment, the tablet of the invention contains one (or more) pH adjusters. In general, acids or buffer substances are suitable as pH adjusters.

The acids used are usually organic acids with a pKs value of 2 to 6, preferably 3 to 5, which have a water solubility of >1 g/250 ml at 20° C., preferably >1 g/160 ml at 25° C. Examples are fumaric acid, tartaric acid, citric acid, malic acid, glutamic acid, aspartic acid and mixtures thereof.

“Buffer substances” are understood to mean mixtures of substances whose pH value in aqueous solutions is substantially insensitive to small amounts of acidic or base additives. Equimolecular mixtures of weak acids and their alkaline salts are particularly suitable for this purpose. The same applies analogously to the bases. Suitable buffer substances are, for example, an acetate buffer, citrate or phosphate buffer. The acetate buffer is preferably a mixture of CH₃COOH and CH₃COOM. The phosphate buffer is preferably a mixture of H₂PO₄M and HPO₄M₂. M is an alkali metal, preferably sodium. The acetate buffer is particularly preferred.

The pH adjusters are usually selected such that a pH value of 3.5 to 5.5, preferably 4 to 5, can be adjusted. The acetate buffer is particularly preferred as the pH adjuster.

In this embodiment, the tablet of the invention usually contains 0.1 to 15% by weight pH adjuster, preferably 0.5 to 10% by weight, more preferably 1 to 8% by weight, based on the total weight of the tablet.

The use of a pH adjuster contributes especially to allowing the tablet of the invention to be administered independently of mealtimes. In addition, the use of the pH adjuster leads to an improvement in solubility. The inventors of the present application have also established that this effect can also occur independently of any compacting.

The subject matter of the invention is therefore an oral dosage form containing cinacalcet and a pH adjuster. The pH adjuster is preferably suitable for adjusting a pH value of 3.5 to 5.5, preferably 4 to 5. Administration occurs especially independently of mealtimes. The expression “administration independently of mealtimes” is understood to mean that the patient may take the drug with meals, but does not necessarily have to take it at mealtimes.

Another subject matter of the invention is the use of a pH adjuster which is suitable for adjusting a pH value of 3.5 to 5.5, preferably 4 to 5, for preparing a pharmaceutical formulation containing cinacalcet as the active agent.

The oral dosage form is, for example, capsules, powder or granules for filling in sachets or tablets. Tablets are preferred.

The above statements on the pH adjusters apply to these oral dosage forms of the invention and the use of the invention.

The tablet of the invention preferably contains fillers. “Fillers” are generally understood to mean substances which serve to form the body of the tablet in the case of tablets with small amounts of active agent (e.g. less than 70% by weight). This means that fillers “dilute” the active agents in order to produce an adequate tablet-compression mixture. The usual purpose of fillers, therefore, is to obtain a suitable tablet size.

Examples of preferred fillers are lactose, lactose derivatives, starch, starch derivatives, treated starch, chitin, cellulose and derivatives thereof, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride. Prosolv® (microcrystalline cellulose modified with SiO₂, Rettenmaier & Söhne, Germany) can also be used.

Other fillers that can be used are sugar alcohols and/or disaccharides, such as mannitol, sorbitol, xylitol, isomalt, glucose, fructose, maltose and mixtures thereof. The term “sugar alcohols” in this context also includes monosaccharides.

Fillers are generally used in an amount of 1 to 80% by weight, more preferably 30 to 60% by weight, especially 20 to 40% by weight, based on the total weight of the formulation.

The tablet of the invention may also contain additives to improve powder flowability. One example of an additive to improve powder flowability is disperse silica, e.g. known under the trade name Aerosil®. Preferably, silica is used with a specific surface area of 50 to 400 m²/g, determined by gas adsorption in accordance with Ph. Eur., 6th edition 2.9.26.

Additives to improve powder flowability are generally used in an amount of 0.1 to 5% by weight, e.g. 1.5 to 4% by weight, based on the total weight of the formulation.

Lubricants can be used in addition. Lubricants are generally used in order to reduce sliding friction. In particular the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium steparyl fumarate (Pruv®) and/or magnesium stearate.

Lubricants are generally used in an amount of 0.1 to 5% by weight, preferably 1.0 to 3% by weight, based on the total weight of the formulation.

Anti-stick agents can be used in addition. “Anti-stick agents” are usually understood to mean substances which reduce agglomeration in the core bed. Examples are talcum, silica gel, polyethylene glycol (preferably with 2,000 to 10,000 g/mol weight-average molecular weight) and/or glycerin monostearate.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. Sorbitol, for example—if used as a filler—is not also counted as a hydrophilising agent in addition. Similarly, microcrystalline cellulose—if used as a hydrophilising agent—is not additionally used as a disintegrant, for example (even though microcrystalline cellulose also exhibits a certain disintegrating effect).

In a preferred embodiment, the tablet of the invention contains the following ingredients (based on the total weight of the tablet core):

15 to 40% by weight cinacalcet
15 to 35% by weight hydrophilising agent
15 to 40% by weight filler
15 to 35% by weight disintegrant and
1 to 4% by weight lubricant.

In an alternative preferred embodiment, the tablet of the invention contains the following ingredients (based on the total weight of the tablet core):

more than 40 to 60% by weight cinacalcet
15 to 35% by weight hydrophilising agent
0 to 10% by weight filler
15 to 35% by weight disintegrant and
1 to 4% by weight lubricant.

The tablets of the invention preferably do not contain any polymers that lead to a delayed release. It is especially preferable for the tablets of the invention not to contain any polymers that have a molecular weight of more than 150,000 g/mol.

Another subject matter of the invention is a method of preparing the tablets of the invention, comprising the steps of

(a) mixing (i) crystalline cinacalcet or its pharmaceutically acceptable salts with (ii) a hydrophilising agent and optionally further pharmaceutical excipients;
(b) compacting it into flakes;
(c) granulating the flakes;
(d) compressing the resulting granules into tablets, optionally with the addition of further pharmaceutical excipients; and
(e) optionally film-coating the tablets.

In principle, all the explanations given above on preferred embodiments of the intermediate of the invention also apply to the method of the invention.

In a preferred embodiment, in step (a) of the method of the invention (i) crystalline cinacalcet or its pharmaceutically acceptable salts are mixed with (ii) a hydrophilising agent and optionally further pharmaceutical excipients (β)—as described above.

As mentioned above, the hydrophilising agent preferably does not include any polymer with a weight-average molecular weight of more than 150,000 g/mol. The same applies to the pharmaceutical excipients added in step (a) (and/or also in step (d)) of the method of the invention.

The mixing can be performed in conventional mixers. Alternatively, the mixing of the active agent and excipients can also be performed after the granulation step (c). As a further alternative, it is equally possible that the intermediate of the invention is mixed with part of the excipients (e.g. 50 to 95%) before compacting (b), and that the remaining part of the excipients is added after the granulation step (c). In the case of multiple compacting, the excipients should preferably be mixed in before the first compacting step, between multiple compacting steps or after the last granulation step.

In a preferred embodiment, in step (a)

100% of the cinacalcet used,
100% of the hydrophilising agent used,
optionally 20 to 70% of the filler used and
optionally 30 to 70% of the disintegrant used and
optionally 10 to 40% of the lubricant used
are mixed. The remaining optional amounts of filler, disintegrant and lubricant are optionally added subsequently in step (d).

In step (b) of the method of the invention, the mixture from step (i) is compacted into the intermediate of the invention. It is preferable here that it should be dry compacting (i.e. the compacting is preferably performed in the absence of solvents, especially in the absence of organic solvents).

The compacting conditions in step (b) are preferably selected such that the flakes have a density of 0.8 to 1.3 g/cm³, preferably 0.9 to 1.20 g/cm³, especially 1.01 to 1.15 g/cm³.

The term “density” here preferably relates to the “pure density” and is determined as described above.

The compacting is preferably carried out in a roll granulator.

The rolling force is usually 5 to 70 kN/cm, preferably 10 to 60 kN/cm, more preferably 15 to 50 kN/cm, especially 15 to 25 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 55° C.

In step (c) of the method of the invention, the flakes are granulated. The granulation can be performed with methods known in the state of the art.

In a preferred embodiment, the granulation conditions are selected such that the resulting particles (granules) have a volume-average particle size (D50) value) of 75 to 600 μm, more preferably 120 to 500 μm, even more preferably 150 to 400 μm, especially 200 to 350 μm. The volume-average particle size is determined (as described above) by means of laser diffractometry (using a Malvern Instruments Mastersizer 2000). In an alternative embodiment, the granulation conditions are selected such that the resulting particles (granules) have a weight-average particle size (D50) value) of 75 to 600 μm, more preferably 120 to 500 μm, even more preferably 150 to 400 μm, especially 200 to 350 μm. The weight-average particle size is determined by means of screen analysis (using a Retsch® AS 2000, amplitude 1.5 sec., interval 10 min., amount of sample 15.8 g).

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

A Comil® U5 (Quadro Engineering, USA) is, for example, used for granulating.

In addition, the granulation conditions are preferably selected such that the resulting granules have a bulk density of 0.3 to 0.85 g/ml, more preferably 0.4 to 0.8 g/ml, especially 0.5 to 0.7 g/ml. The Hausner factor is usually in the range from 1.02 to 1.3, more preferably from 1.03 to 1.25 and especially from 1.04 to 1.15. The “Hausner factor” in this context means the ratio of tapped density to bulk density. The bulk density and tapped density are determined in accordance with USP 24, test 616 “Bulk Density and Tapped Density”.

It may happen that the intermediates of the invention do not have a sufficiently rough surface, so that the compacting step (ii) described above is rendered more difficult. Therefore, depending on the nature of the surface, the compacting step (b) and the granulation step (c) may be repeated if necessary.

In a preferred embodiment, the method of the invention is therefore adapted such that multiple compacting occurs, with the granules resulting from step (c) being returned one or more times to the compacting step (b).

The granules from step (c) are preferably returned 1 to 5 times, especially 2 to 3 times.

In step (d) of the method of the invention, the granules obtained in step (c) are pressed into tablets, i.e. the step involves compression into tablets. The compression can be performed with tableting machines known in the state of the art, such as eccentric presses or rotary presses. In the case of rotary presses, a compressive force of 2 to 40 kN, preferably 2.5 to 35 kN, is usually applied. As an example, the Fette® 102i press (Fette GmbH, Germany) is used. In the case of eccentric presses, a compressive force of 1 to 20 kN, preferably 2.5 to 10 kN, is usually applied. By way of example, the Korsch® EK0 is used.

Process step (d) is preferably performed in the absence of solvents, especially organic solvents, i.e. as dry compression.

In step (d) of the method of the invention, pharmaceutical excipients (β) may be added to the granules from step (c). On this subject, reference may be made to the above explanations on suitable excipients.

The subject matter of the invention comprises not only the method of the invention, but also the tablets produced with this method. It has been found that the tablets produced with this method preferably have a bimodal pore size distribution. Hence, the subject matter of the invention comprises tablets containing cinacalcet or a pharmaceutically acceptable salt thereof, preferably in crystalline form, especially in the form of the intermediate of the invention, and optionally pharmaceutically acceptable excipients, wherein the tablets have a bimodal pore size distribution.

This tablet of the invention is formed when the granules from process step (c) are compressed. This compressed material consists of solid and pores. The pore structure can be characterised more specifically by determining the pore size distribution.

The pore size distribution was determined by means of mercury porosimetry. Mercury porosimetry measurements were made with the Micromeritics, Norcross, USA, “Poresizer” porosimeter. The pore sizes were calculated assuming a mercury surface tension of 485 mN/m. The cumulative pore volume was used to calculate the pore size distribution as the cumulative frequency distribution or proportion of the pore fractions in percent. The average pore diameter (4V/A) was determined from the total specific mercury intrusion volum (Vgesᵢₙₜ) and the total pore surface area (Agesₚₒᵣ) according to the following equation.

$$4 V / A = \frac{4 \cdot {Vges}_{int} [ ml \text{/} g ]}{{Ages}_{por} [ {m}^{2} \text{/} g ]}$$

“Bimodal pore size distribution” is understood to mean that the pore size distribution has two maxima.

One maximum of the pore size distribution is preferably at a pore size of 0.01 to 0.4 μm, more preferably at 0.05 to 0.3 μm, especially 0.1 to 0.2 μm. A second maximum is preferably at a pore size of 0.4 to 5 μm, more preferably at 0.5 to 2 μm, especially 0.6 to 1.2 μm.

A tablet is especially preferred which, on the one hand, has the pore size distribution explained above and, on the other hand, contains the pH adjuster explained above. These tablets are especially suitable for administration independently of meals.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer in step (e) of the method of the invention. The above-mentioned ratios of active agent to excipient, however, relate to the uncoated tablet.

For film-coating, macromolecular substances are preferably used, such as modified celluloses, polymethacrylates, polyvinyl pyrrolidone, polyvinyl acetate phthalate, zein and/or shellack.

HPMC is preferably used, especially HPMC with a weight-average molecular weight of 10,000 to 150,000 g/mol and/or an average degree of substitution of —OCH₃ groups of 1.2 to 2.0.

The thickness of the coating is preferably 2 to 100 μm.

The tableting conditions are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 70 to 200 N, particularly preferably 100 to 150 N, especially if the tablet weight is more than 200 mg. if the tablet weight is 200 mg or less, the resulting tablets preferably have a hardness of 30 to 100 N, particularly preferably 50 to 70 N. The hardness is determined in accordance with Ph. Eur. 6.0, section 2.9.8.

In addition, the resulting tablets preferably have a friability of less than 3%, particularly preferably less than 1%, especially less than 0.8%. The friability is determined in accordance with Ph. Eur. 6.0, section 2.9.7.

Finally, the tablets of the invention usually have a “content uniformity” of 95 to 105% of the average content, preferably 98 to 102%, especially 99 to 101% of the average content. The “content uniformity” is determined in accordance with Ph. Eur. 6.0, section 2.9.6.

The release profile of the tablets of the invention according to the USP method (paddle, 900 ml 0.1 N HCl, pH 1.2, 37° C., 75 rpm) after 10 minutes usually indicates a content released of at least 30%, preferably at least 50%, especially at least 70%.

The above details regarding hardness, friability, content uniformity and release profile preferably relate here to the non-film-coated tablet.

As an alternative to compression into tablets, the granules resulting in step (c) of the method of the invention may also be processed—optionally with the addition of further pharmaceutical excipients—into a particulate dosage form, such as by filling into capsules or sachets.

Hence, the subject matter of the invention encompasses an oral dosage form containing cinacalcet, hydrophilising agent and disintegrant for the treatment of hyperparathyroidism, wherein the administration is independent of mealtimes. In a preferred embodiment, a disintegrant is used in an amount of 10 to 30% by weight, based on the total weight of the oral dosage form. In a further preferred embodiment, a polyoxyethylene/polyoxypropylene block polymer is used as the hydrophilising agent for this purpose, especially as described in more detail above. In a further preferred embodiment, the content of cinacalcet is 20 to 60% by weight, especially 40 to 60% by weight.

The invention will now be illustrated with reference to the following examples.

### EXAMPLES

### Example 1

The following formulation was used:

### Core:

| | |
|---|---|
| cinacalcet hydrochloride | 33.06 mg |
| (D50 101 μm, water content 0.06%) | |
| polyvinyl pyrrolidone (Mw 30,000) | 10 mg |
| PEG 6000 | 15 mg |
| Prosolv ® (filler) | 50 mg |
| tartaric acid | 60 mg |
| magnesium stearate | 1.8 mg |
| crospovidone | 4 mg |

### Film:

| | |
|---|---|
| HPMC (Mw 100,000) | 1.5 mg |
| titanium dioxide | 0.4 mg |
| colorant | 0.1 mg |

Cinacalcet hydrochloride was compacted into flakes (=intermediate) on a roll compactor with polyvinyl pyrrolidone and PEG 6000 together with 70% by weight of the Prosolv® and crospovidone. After that, the flakes were screened with a screen (Comil U5; 1.0 mm). Then the resulting granules were mixed with the remaining core excipients (Turbula 10B) and compressed into tablets (Fette 102i). That was followed by film-coating the tablet core.

### Example 2

The following formulation was used:

### Core:

| | |
|---|---|
| cinacalcet hydrochloride (D50 101 μm), | 33.0 mg |
| polyoxyethylene/polyoxypropylene | 30.0 mg |
| block polymer (Mw approx. 8,350) | |
| sorbitol (filler) | 38.0 mg |
| sodium stearyl fumarate | 4.00 mg |
| crospovidone | 40.0 mg |

### Film:

Opadry® AMB 6.40 mg

The production process comprised the following steps:
cinacalcet HCl, polyoxyethylene/polyoxypropylene block polymer, 20 mg crospovidone, 20 mg sorbitol and 1.5 mg sodium stearyl fumarate were premixed for 5 min in a Turbula® TBD 10 (free-fall mixer),
the premixture was screened (mesh width 0.315 mm) and mixed for a further 10 minutes,
it was compressed using a Korsch® EKO (eccentric press, 18 kN, 20 mm bi-planar) and granulated by screening (mesh width 0.6 mm),
the granules were mixed for 10 min together with the remaining crospovidone and sorbitol,
sodium stearyl fumarate was added by screening (mesh width 0.3 mm) and mixed for a further 5 minutes,
the mixture obtained was pressed into tablets (9.7×5 r 3.6; 4.5 kN; 50 N), and
the tablets were coated with an Opadry® AMB solution.

The resulting tablets exhibited advantageous solubility properties, which were maintained after storage for three months (at 40° C., 75% air humidity), cf. Example 4.

### Example 3

The following formulation was used:

### Core:

| | |
|---|---|
| cinacalcet hydrochloride (D50 101 μm) | 33.0 mg |
| polyoxyethylene/polyoxypropylene | 16.5 mg |
| block polymer (Mw approx. 8,350) | |
| sodium stearyl fumarate | 3.00 mg |
| crospovidone | 17.0 mg |

### Film:

Opadry® AMB 4.0 mg

The production process comprised the following steps:
cinacalcet HCl, polyoxyethylene/polyoxypropylene block polymer, 9 mg crospovidone and 1 mg sodium stearyl fumarate were mixed for 5 min in a Turbula® TBD 10 (free-fall mixer),
the premixture was screened (mesh width 0.6 mm) and mixed for a further 10 minutes,
it was compressed using a Korsch® EKO (eccentric press, 18 kN, 20 mm bi-planar) and granulated by screening (mesh width 0.6 mm),
the granules were mixed for 10 min together with the remaining crospovidone,
sodium stearyl fumarate was added by screening (mesh width 0.3 mm) and mixed for a further 5 minutes,
the mixture obtained was pressed into tablets (6 mm r 7.5, 5 kN, 50 N), and
the tablets were coated with an Opadry® AMB solution.

The resulting tablets exhibited advantageous solubility properties (>80% after 15 min, >95% after 30 min), which were maintained after storage for three months (40° C., 75% air humidity).

### Comparative Example 1

For comparison purposes, tablets in accordance with WO 2005/34928 A 1 (paragraph [0057]), containing 30 mg micronised cinacalcet HCl, were produced by means of wet granulation. The solubility behaviour was investigated in Example 4.

### Example 4

The in-vitro solubility behaviour of (non-film-coated) tablets in accordance with Example 2 and Comparative example 1 was investigated in accordance with USP (paddle, 900 ml 0.1 N HCl, pH 1.2, 37° C., 75 rpm) before and after storage (40° C., 75% rel. air humidity).

| | | Amount | Amount | Amount | Amount | Amount |
|---|---|---|---|---|---|---|
| | Stirring | without | after | after | after | after |
| Example | time | storage | 2 weeks | 4 weeks | 8 weeks | 12 weeks |
| Example 2 | 15 | 96.4 | 101.7 | 101.5 | 103.1 | 101.0 |
| Comparative | 15 | 83.1 | 86.5 | 84.3 | 83.9 | 83.7 |
| example 1: | | | | | | |

The measurement shows that the tablets of the invention exhibit very good solubility behaviour even after storage, and it was also possible to avoid micronisation of the active agent.

### Example 5

Tablets in accordance with Example 2 were investigated before and after storage (40° C., 75% rel. air humidity) to determine the contents and measure the impurities by means of the HPLC method.

HPLC parameters:
column: X-Bridge C18 150×4.6 mm, 3.5 μm,
flow rate: 0.9 ml/min.
column temperature: 60° C.,
injection volume: 2 μl,
eluant A: 25 mmol/l KH₂PO₄*H₂O pH 3.00±0.05
eluant B: acetonitrile

| | | |
|---|---|---|
| pump gradient: | time [min] | % B |

| | |
|---|---|
| 0 | 25 |
| 3 | 25 |
| 22 | 65 |
| 25 | 25 |

wavelength: 225 nm,
sample solvent: water/acetonitrile 50/50
sample concentration: 450 μg/ml

| | without | after | after |
|---|---|---|---|
| | storage | 4 weeks | 12 weeks |
| content | 97.2 | 96.83 | 99.27 |
| total impurities | 0.03 | 0.06 | 0.08 |

The analysis shows that the tablets of the invention exhibit very good storage stability.

1. An intermediate, obtainable by jointly compacting
(i) crystalline cinacalcet or a pharmaceutically acceptable salt thereof, with
(ii) a hydrophilising agent.

2. The intermediate as claimed in claim 1, wherein the compacting is performed in a roller compacter and the rolling force is 5 to 70 kN/cm, preferably 10 to 50 kN/cm.

3. The intermediate as claimed in claim 1, wherein the density of the intermediate is 0.8 to 1.3 g/cm³, preferably 0.9 to 1.20 g/cm³.

4. The intermediate as claimed in claim 1, wherein crystalline cinacalcet or a pharmaceutically acceptable salt thereof with a specific surface area of 0.01 to 8 m²/g is used.

5. The intermediate as claimed in claim 1, wherein hydrophilic polymers with a weight-average molecular weight of less than 150,000 g/mol are used as the hydrophilising agent.

6. The intermediate as claimed in claim 5, wherein polyvinyl pyrrolidone, a copolymer of vinyl pyrrolidone and vinyl acetate and/or polyethylene glycol is used as the hydrophilising agent.

7. The intermediate as claimed in claim 1, wherein the weight ratio of component (i) to component (ii) is 1:5 to 5:1.

8. An immediate-release tablet containing
(α) an intermediate in accordance with claim 1 and
(β) pharmaceutical excipients.

9. The tablet as claimed in claim 8, characterised in that component (β) contains disintegrant.

10. The tablet as claimed in claim 9, wherein the disintegrant is present in an amount of 10 to 30% by weight, based on the total weight of the formulation.

11. The tablet as claimed in claim 8, wherein the tablet has a cinacalcet content of 40 to 60% by weight.

12. A tablet containing crystalline cinacalcet or its pharmaceutically acceptable salts and a hydrophilising agent, wherein the tablet has a bimodal pore distribution.

13. The method of preparing a tablet as claimed in claim 8 comprising the steps of
(a) mixing (i) crystalline cinacalcet or its pharmaceutically acceptable salts with (ii) a hydrophilising agent and optionally further pharmaceutical excipients;
(b) compacting it into flakes;
(c) granulating the flakes;
(d) compressing the resulting granules into tablets, optionally with the addition of further pharmaceutical excipients; and
(e) optionally film-coating the tablets.

14. The use of a pH adjuster which is suitable for adjusting a pH value of 3.5 to 5.5, preferably 4 to 5, for preparing a pharmaceutical formulation containing cinacalcet as the active agent.

15. An oral dosage form containing cinacalcet, hydrophilising agent and disintegrant for the treatment of hyperparathyroidism, wherein the administration is independent of mealtimes.
